(19) United States
(12) Patent Application Publication
(10) Pub. No.: US 2008/0286216 A1
(43) Pub. Date: Nov. 20, 2008

(54) Surface Active Calcium Phosphates

(76) Inventors: David Kannar, Belgrave South VIC (AU); Robert Verdicchio, Succasunna, NJ (US)

Correspondence Address: HAHN LOESER & PARKS, LLP, One GOJO Plaza, Suite 300, AKRON, OH, 44311-1076, US

(21) Appl. No.: 12/095,044

(22) PCT Filed: Dec. 15, 2006
(86) PCT No.: PCT/AU2006/001914

§ 371 Date: May 27, 2008

Related U.S. Application Data
(60) Provisional application No. 60/751,335, filed on Dec. 16, 2005.

(51) Int. Cl. A61K 8/55; C07F 9/06; A61Q 11/00

(52) U.S. Cl. 424/57; 564/12

(57) ABSTRACT

A nitrogen complex of an inorganic phosphate comprising the anionic sub-structure (I); whereby a nitrogen atom (N) of a complexing agent is complexed to an oxygen atom (O) of the inorganic phosphate; wherein the nitrogen atom is selected from the group consisting of a nitrogen atom of an amine group or a nitrogen atom of a N-heteroaromatic ring; and wherein A and A′ are each independently selected from the group of constituents consisting of a hydroxyl group (—OH), an oxide group (—O⁻), the group —OR where R is alkyl or substituted alkyl; an oxygen to which a nitrogen is complexed (—0←N) wherein the nitrogen atom is selected from the group consisting of a nitrogen atom of an amine group or a nitrogen atom of a N-heteroaromatic ring; and a phosphate of the form (i); wherein each of A″ and A′″ are selected independently from the same group of substituents as A and A′; wherein at least one of A, A′, A″ and A′″ is an oxide (—O⁻) with the proviso that when A′ and A′″ are not present then at least one of A and A′ is an oxide (—O⁻).

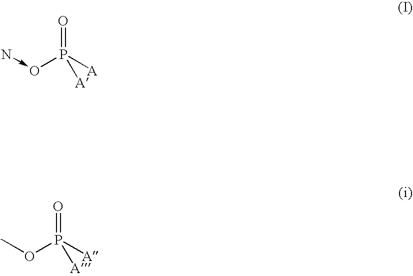

### FIELD OF THE INVENTION

This invention relates to novel complexes of inorganic phosphates, particularly calcium phosphates, and the use of said complexes in the treatment of caries in teeth.

### BACKGROUND OF THE INVENTION

In this specification where a document, act or item of knowledge is referred to or discussed, this reference or discussion is not an admission that the document, act or item of knowledge or any combination thereof was at the priority date publicly available, known to the public, part of the common general knowledge, or known to be relevant to an attempt to solve any problem with which this specification is concerned.

While the cause of dental caries (or tooth decay) can be multifactorial, the role of indigenous streptococci in the development of the disease is well established. Streptococcus mutans and other cariogenic bacteria including Lactobacilli sp., produce water-insoluble glucan using dietary sucrose as a nutrient and glycosyltransferase as an enzyme. Glucan covers the tooth surface, resulting in dental plaque. While in this matrix, bacteria ferment the sucrose from food producing high concentrations of acid that demineralise the adjoining tooth enamel creating tooth decay. Damage to tooth enamel is followed by decomposition of the underlying dentine and cementum. If left to accumulate in gingival crevices (between the teeth and gingivae), micro-organisms can cause soft tissue damage and resorption of bone, which commonly occurs in periodontal disease.

Despite a substantial decline in prevalence and severity of dental caries during the 20th century, incidence of this disease remains a major public health problem. 67% of persons aged 12-17 years and 94% of persons aged at least 18 years are reported to have experienced caries in their permanent teeth. Dental caries is therefore still a major public health problem, particularly in ethnic and lower socio-economic groups.

Various anti-cariogenic agents are known and are discussed below.

In 1962, the American Dental Association recommended supplementation of 0.7-1.2 ppm fluoride in drinking water. Since the introduction of this regime, the incidence of dental caries has been substantially reduced.

In 2000, the British Medical Research Council funded a large study to review the use of fluoride in drinking water as a public health measure. The report concluded that although fluoridation remains an effective public health measure, the incidence of fluorosis may be as high as 48% in people living in fluoridated areas and at least 12.5% experienced aesthetically unacceptable changes to the appearance of their teeth. Prevalence of dental fluorosis in the United States of America is reported to have increased in both optimally fluoridated and non-fluoridated areas. This is thought to be due to an increase in the fluoride level of food and beverages through processing with fluoridated water, inadvertent ingestion of fluoride toothpaste, and the inappropriate use of dietary supplements. As a result, public support for water fluoridation is waning, yet there is an increasingly apparent expectation that most teeth will be retained for a lifetime. Clearly, there is an urgent need for alternative agents and strategies for reducing caries. A non-toxic compound that could reduce the dose of fluoride for treating dental caries is desirable.

The surface of a tooth is made of a crystalline material termed enamel which comprises impure forms of hydroxyapatite [Ca₁₀(PO₄)₆(OH)₂]. Organic acid secreted by bacteria in dental plaque can dissolve the calcium and phosphate of the enamel and dentin (the hard tissue beneath the surface of the enamel) in a process called demineralization. When the plaque is buffered by saliva, pH is returned to neutral, and calcium and phosphate ions in saliva are reincorporated into the dentin through the plaque (remineralization). The balance between demineralization and remineralization depends largely on the oral environment, particularly, the pH of the saliva and dental plaque, and the concentrations of calcium and phosphate.

In recent years, research has demonstrated that some anticaries agents not only prevent dental caries by making the enamel more acid resistant but also by (1) enhancing remineralization, and (2) inhibiting bacterial activity in dental plaque. Enhancing remineralization and reducing cariogenic bacteria have therefore become an important initiative and strategy to reduce incidence of dental caries.

Sugar alcohols (such as xylitol, mannitol, galactitol, palatinit and inositol) are known as anti-dental caries agents (Japanese Publication No. 2000-128752 and Japanese Publication No. 2000-53549, Japanese Publication No.2000-281550) because they are poor bacterial substrates inhibiting plaque formation and subsequent production of water-insoluble glucan and organic acids (S. Hamada et al., J. Jpn. Soc. Starch Sci., 1981;31:83-91). The compounds are however only effective at high concentrations and large intakes of sugar alcohols can loosen bowel motions, which is not desirable.

Sugar alcohols and phosphorylated oligosaccharides are also known to promote remineralization of teeth (Japanese Publication No. 11-12143). When combined with fluorine or zinc, remineralization of teeth is promoted (Japanese Publication No. 2000-247852). Importantly, in these disclosures the oligosaccahrides are phosphorylated through hydroxyl functional groups.

Fluorine is known in the art to be effective for remineralization of teeth when used at 2 ppm. Fluorine is incorporated into the hydroxyapatite crystal, which is then converted to a hard crystal structure resistant to demineralization. Use of fluorine in this manner has been proposed in various oral compositions. Japanese Publication No. 11-130643 discloses an oral composition containing calcium carbonate and fluoride. Combination of fluoride with sugar alcohol is also taught to enhance the ability of fluorine to remineralise teeth (Japanese Publication No. 11-21217, Japanese Publication No.2000-72638, and Japanese Publication No. 2000-154127).

It is known in the art that application of calcium phosphate to teeth promotes remineralization (Japanese Publication No.11-228369 and Japanese Publication No. 10-310513). Japanese Publication No. 11-29454 discloses an oral composition containing calcium carbonate and alginate. The inventors teach that the composition enhances the ability of calcium carbonate to adhere to teeth and improve neutralization of pH and subsequent remineralization.

In 1985, Onisi reported the feasibility of instituting a tea-drinking program in Japanese schools to reduce incidence of dental caries in children and enable reduction of fluoride supplementation with associated fluorosis. Caries reduction rates resulting from regular tea consumption in school children tested ranged from 22.1 to 26.1%. Anti-cariogenic effects of tea have since been attributed to polyphenolic compounds. Addition of these compounds to dental hygiene products and sucrose containing foods has been considered as a method of inhibiting tooth demineralization but has not been commercially undertaken. In any case, polyphenolic compounds are bitter, and use at high concentrations may interfere with taste.

Caseinate (phosphoprotein salt of cow's milk) is known to be anticariogenic when added to drinking water of rats. U.S. Pat. No. 5,130,123 describes the anticariogenic use of Caseinate, but the compounds are a bitter and unpalatable ingredient when used in therapeutically useful doses, even when formulated with chocolate confectionary. Lower levels of Caseinate do not significantly improve the confection's anticariogenic activity.

In U.S. Pat. No. 5,015,628, Reynolds discloses a method of reducing tooth demineralisation using a topically applied trypsin digest of milk Caseinate. In U.S. Pat. No. 5,227,154, Reynolds teaches that casein phosphopeptide complexes stabilize calcium phosphate and facilitate incorporation and accumulation of calcium and other remineralising or antibacterial ions in dental plaque. The accumulation of calcium phosphate ions in plaque is thought to slow demineralisation and is described as anticariogenic.

In WO 02067871, Kenji describes use of buffering agents to restore oral pH to neutral. At neutral pH, calcium and phosphate ions in saliva are reincorporated into dentin through the plaque and tooth remineralization is promoted. Although Kenji describes the use of surfactants in toothpastes and dentrifices, the authors do not describe the use of complexes to improve the substantivity of the buffering agents to tooth enamel surfaces. Kenji does not teach that surfactants improve the substantivity of the buffering agents or disclose the use of amine or quaternary amine complexes.

In EP 0968700, Dimitri discloses the use of ion-exchange resins, cationic and anionic, charged with Ca²⁺, F⁻ and PO₄³⁻ions, in an approximate molar ratio of 2:1:1, to remineralise tooth enamel. The preferred resins are those whose base is cross-linked polystyrene with 2-14% divinylbenzene. The material is useful as a first filler in the treatment of caries, especially deep caries, leading to remineralization of the dentin with a composition very close to the original composition. It is also useful as a component of dentifrice products such as pastes, elixirs and dental floss.

Numerous patents and publications describe chewing gums containing compounds of calcium, such as calcium lactate (DE Pat. No. 2,543,489), calcium nitrate (WO 97/06774); of fluorides (Santos de los, R. et al., Caries Res., 1994;28(6):441-446, Wang, C W, et al., Caries Res., 1993;27(6):455-460, Lamb W J. et al., Caries Res., 1993;27(2):111-116); or of phosphate, such as potassium phosphates (WO 97/06774, U.S. Pat. No. 5,958,380), sodium phosphates (DE Pat. No. 2,543,489), calcium phosphates (WO 98/07448) or of calcium, phosphate and fluoride (U.S. Pat. No. 5,460,803) and encapsulated ion-exchange resins to remineralise teeth (WO 02/49448 Gonzalo). Use of calcium phosphate sterol complexes to improve substantivity, anticariogenic or remineralizing effect is not described in the background art.

### SUMMARY OF THE INVENTION

The present invention describes the preparation of novel stable inorganic phosphate complexes. These complexes can be used in treating the teeth with a composition containing the complexes. It has surprisingly been found that a composition comprising the complexes of the present invention facilitates tooth remineralisation and reduces the incidence of dental caries.

According to a first aspect of the invention, there is provided a nitrogen complex of an inorganic phosphate comprising the anionic sub-structure (I)

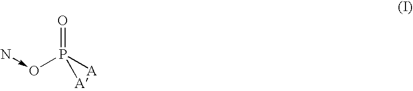

whereby a nitrogen atom (N) of a complexing agent is complexed to an oxygen atom (O) of the inorganic phosphate;
wherein the nitrogen atom is selected from the group consisting of a nitrogen atom of an amine group or a nitrogen atom of a N-heteroaromatic ring; and
wherein A and A′ are each independently selected from the group consisting of a hydroxyl group (—OH), an oxide group (—O⁻), the group —OR where R is alkyl or substituted alkyl; an oxygen to which a nitrogen is complexed (—O←N) wherein the nitrogen atom is selected from the group consisting of a nitrogen atom of an amine group or a nitrogen atom of a N-heteroaromatic ring; and a phosphate of the form

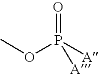

wherein each of A″ and A′″ are selected independently from the same group of substituents as A′ and A″;
wherein at least one of A, A′, A″ and A′″ is an oxide (—O⁻) with the proviso that when A″ and A′″ are not present then at least one of A and A″ is an oxide (—O⁻).

The counter cation is preferably chosen from the group consisting of alkali metals (Group I) and alkaline earth metals (Group II).

According to a second aspect of the invention, there is provided a method of preparing a complex according to the first aspect comprising the step of reacting a complexing agent containing nitrogen with an inorganic phosphate.

According to a third aspect of the present invention, there is provided a composition for the administration of an inorganic phosphate, the composition comprising an effective amount of one or more complexes of the first aspect.

According to a fourth aspect of the present invention, there is provided a method of treating dental caries comprising the step of administering a complex of the first aspect or a composition of the third aspect to the mouth or teeth.

### DETAILED DESCRIPTION

The present invention describes the preparation of stable complexes of inorganic phosphates, particularly calcium phosphates, by their reaction with complexing agents comprising nitrogen. Such inorganic phosphate complexes may be used in the treatment of caries by administering a composition containing the complexes to the teeth. It has surprisingly been found that a composition comprising such complexes of inorganic phosphates facilitates tooth remineralization and reduces the incidence of dental caries.

According to a first aspect of the invention, there is provided a nitrogen complex of an inorganic phosphate comprising the anionic sub-structure (I)

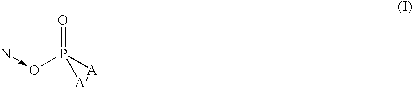

whereby a nitrogen atom (N) of a complexing agent is complexed to an oxygen atom (O) of the inorganic phosphate;
wherein the nitrogen atom is selected from the group consisting of a nitrogen atom of an amine group or a nitrogen atom of a N-heteroaromatic ring; and
wherein A and A′ are each independently selected from the group of constituents consisting of a hydroxyl group (—OH), an oxide group (—O⁻); the group —OR where R is alkyl or substituted alkyl; an oxygen to which a nitrogen is complexed (—O←N) wherein the nitrogen atom is selected from the group consisting of a nitrogen atom of an amine group or a nitrogen atom of a N-heteroaromatic ring; and a phosphate of the form

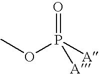

wherein each of A″ and A′″ are selected independently from the same group of substituents as A′ and A″;
wherein at least one of A, A′, A″ and A′″ is an oxide (—O⁻) with the proviso that when A″ and A′″ are not present then at least one of A and A′ is an oxide (—O⁻).

It would be clear to a person skilled in the art that the complexes of the present invention would bear a negative charge due to the presence of the oxide (—O⁻). The counter cation is preferably chosen from the group consisting of alkali metals (Group I) and alkaline earth metals (Group II). More preferably, the counter cation is Ca²⁺.

The preferred inorganic phosphate is selected from the group consisting of calcium phosphates. More preferably, the inorganic phosphate is selected from the group consisting of calcium phosphate monobasic (Ca(H₂PO₄)₂), calcium dibasic (Ca HPO₄), calcium phosphate tribasic (Ca₃(PO₄)₂), superphosphates of calcium, fluorinated calcium superphosphate, calcium phosphate salts either in amorphous or crystalline forms (including apatites and hydroxyapatites) and mixtures thereof.

Preferably, the group R when present is glycerol.

Where the nitrogen is the nitrogen of an amine, the amine may be a primary, secondary, tertiary or quaternary amine. Preferably, the amine is a tertiary amine.

In a preferred form, the amine forms part of a complexing agent of formula (II)

NR¹R²R³ (II)

wherein R¹ is chosen from the group of substituents consisting of straight or branched chain mixed alkyl radicals from C6 to C22 and carbonyl derivatives thereof; R² and R³ are chosen independently from the group of constituents consisting of H, CH₂COOX, CH₂CHOHCH₂SO₃X, CH₂CHOHCH₂OPO₃X, CH₂CH₂COOX, CH₂COOX, CH₂CH₂CHOHCH₂SO₃X or CH₂CH₂CHOHCH₂OPO₃X and X is H, Na, K or alkanolamine provided R² and R³ are not both H; and wherein when R¹ is RCO then R² may be CH₃ and R³ may be (CH₂CH₂)N(C₂H₄OH)—H₂CHOPO₃ or R² and R³ together may be N(CH₂)₂N(C₂H₄)H)CH₂COO—.

A particularly preferred complexing agent is stearamidopropyldimethylamine.

Other complexing agents containing amine groups or other nitrogen functional groups will also be suitable. For instance, amino acids such as arginine, lysine, glycine and histidine; and proteins which are formed from a mixture of amino acids which are joined by a peptide link CO—NH, such as water soluble albumins, insoluble globulins which are soluble in dilute electrolyte solutions, strongly basic protamines of low molecular weight containing high levels of arginine; prolamines, glutelins, sleroproteins such as collagen and phosphoproteins such as casein; lipoproproteins, and clycoproteins also known as mucoproteins containing poly saccharides. Other examples include peptides formed from the hydrolysis of proteins or synthesized directly such glycylglycine. The peptides are defined by the number of amino acids linked to the peptide bond CO—NH—, thus polypeptides in some cases are synonymous with proteins having a molecular weight in the range from 5000 to 6,000,000. Although the dividing line between a protein and polypeptide is unclear, the latter can range from 132.12 as in glycylglycine to 6000 for the purpose of this invention. Also suitable are amine functional sterols and phospholipids containing amine functional groups such as lecithin.

Water soluble polymers having nitrogen with a positive charge have also been found to be suitable complexing agents. The polymer may be amphoteric, zwitterionic, or cationic. Preferred complexing agents of this type include merquats. Merquats are water soluble cationic polymers with a quaternary ammonium functional group on the polymer backbone. Examples of other cationic polymers include the polymer manufactured under the trade name “Ucare JR” by Union Carbide, the cationic polymer manufactured under the trade name “Gafquat” by ISP and cationic Guar Gums sold under the trade name Jaguar.

Nitrogen containing silicone polymers that bear amine groups are also suitable complexing agents. For instance, the aminated polysilicone trimethylsilylamodimethicone has been found by the present inventors to be a suitable complexing agent. The functional nitrogen group can be tertiary or quaternary. Nitrogen containing amphoteric silicone polymers such as those sold as ABIL by Goldschmidt/Degussa fall within this group.

Other suitable complexing agents include cationic, zwitterionic and amphoteric surfactants such as phosphobetaines. The phosphobetaines described in U.S. Pat. Nos. 4,382,046, 4,380,637, 4,261,911, 4,215,064 and 6,180,806 are particularly useful for preparing complexes according to the invention. The remineralization of teeth is particularly enhanced using alkoxylated, and more preferably ethoxylated, adducts of the latter having a zwitterionic cationic charge in the molecule as shown by the structure below:

R⁴—C(O)—O—(CH₂—CH₂—O)ₓ—PO₃—CH₂CHOHCH₂—N⁺—(CH₃)₃—R⁵

where R⁴ and R⁵ are alkyl or mixed alkyl groups having 8 to 22 carbon atoms and x is an integer from 1-500, preferably 4-25. Examples of these compounds are sold commercially by Phoenix Chemical Company, Somerville N.J., under the trade name EPB. Also suitable are the APB phosphobetaines sold by Phoenix Chemical Co having the following structure:

R⁶—C(O)—NH—(CH₂)₃—N⁺(CH₃)₂—CH₂—CH(OH)CH₂-0-PO₃⁼2Na⁺

where R⁶ is alkyl or mixed alkyl groups from C8 to C22.

It is preferred that the complexes exhibit a high level of substantivity so that the complexes will be likely to remain in proximity to the desired administration site subsequent to administration.

In order to more clearly define the invention a number of representative complexes are presented below:

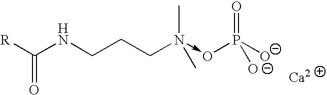

Stearamidopropyldimethylamine calcium phosphate complex (R═C₁₇ alkyl)

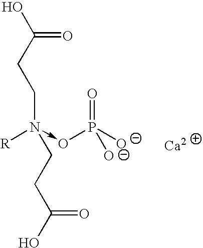

Lauryliminodipropionic acid calcium phosphate complex (R═C₁₁ alkyl)

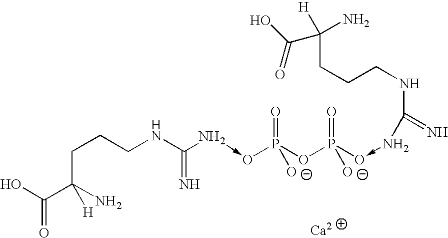

### Arginine₂ Calcium Pyrophosphate Complex

Other potential complexing agents may be found in international patent application no WO02/40034, the disclosure of which is hereby incorporated by reference.

According to a second aspect of the invention, there is provided a method of preparing a complex according to the first aspect comprising the step of reacting a complexing agent containing nitrogen with an inorganic phosphate.

The inorganic phosphate is preferably a calcium phosphate.

According to a third aspect of the present invention, there is provided a composition for administration of an inorganic phosphate comprising an effective amount of one or more complexes of the first aspect.

The term “effective amount” is used herein to refer to an amount that, when the composition is administered in the treatment of a symptom, is sufficient to reach the target site in a human or animal and be measurably effective in the reduction of the symptom. In one preferred embodiment, the symptom is dental caries.

It would be understood by a person skilled in the art that the correct amount of complex to be administered in order to be effective will be variable and dependent on the needs of the afflicted human or animal. Correct dosage should be determined by monitoring individual responses and may be administered over a period of minutes, hours or days, depending upon the concentration of the complex in the composition.

The concentration of the complex in the composition is dependent upon the format of administration. In the treatment of dental caries, the composition may be administered by exposing the teeth to a gel comprising a very high concentration of the complex with concentrations of up to 99.5% w/w. When administered in the form of a toothpaste the concentration of the complex composition would preferably be in the range of from 0.1% to 10% w/w. When administered in the form of a chewing gum, the concentration of the complex composition would preferably be in the range up to 10% w/w. When administered as a dental mouthwash, typical concentrations would lie within the range 0.1% to 4% w/w. Accordingly, depending on the form of the composition, the concentration can lie within the range up to 99.5%.

According to a first preferred embodiment, the composition is preferably an “anti-caries” composition. The term “anti-caries” as used herein refers to both functions of preventing dental caries and treating dental caries. The function of treating dental caries means a function of repairing a portion of a tooth which has been lost due to dental caries. The term “anti-dental caries function” as used herein refers to one or more of the following properties: (1) a pH buffering ability to prevent pH reduction due to acids produced by oral bacteria; (2) an ability to prevent oral bacteria from producing insoluble glucan; and (3) an ability to promote remineralization of teeth in early dental caries. Preferably, the anti-caries function has at least one of the above-described properties, and most preferably all of the above-described properties.

The composition of the present invention can stably provide phosphate and calcium to decayed teeth. The teeth supplied with phosphate and calcium are remineralised, so that a portion of a tooth lost due to dental caries is repaired. It is also important that the compositions include complexing agents (surface active agents and/or polymers) which can be complexed with the inorganic phosphate regardless of charge, ie, anionic, cationic or nonionic. Any of the foregoing can be used in cases where the inorganic phosphates are charge sensitive, in which case, it is though that the inorganic phosphate is (1) complexed, (2) solubilized within the amphoteric/surfactant micelles, and (3) carried within the polymer matrix and deposited via coacervation due to a change in electrokinetic effects within the oral mucosa.

It is also possible and within the scope of the present invention for the anti-caries composition to further comprise combinations of compounds disclosed in the prior art for the treatment of teeth or the mouth cavity including, but not limited to, pyrophosphates for treatment of dental calculus, antibacterials, pharmaceuticals, nutrients, fluoride and phosphatase inhibitors such as vinyl ether maleic acid polymers, aggregating divalent and trivalent metal ions; whitening agents such as bicarbonates that increase pH, calcium phosphate monofluorophosphate urea (CPMU) and substances that change oral pH.

Accordingly, the composition of the present invention may further comprise antibacterials such as phenolics, salicylamides, salicylanilides, plant extracts and oils, metal ions such as copper, stannous copper, silver, stannous silver, zinc, stannous zinc, anti-plaque agents, anticaries agents, pH buffering agents, anti-staining agents, bleaching agents, desensitizing agents, dyes, colors, surfactants, binders, sweeteners, humectants, abrasive agents and other additives suitable to improve oral health or formulation of oral health products and suitable for inclusion in dietary compositions, pharmaceutical preparations or dental hygiene products.

A person skilled in the art would know that the composition may further comprise various excipients. The choice of excipients would depend on the characteristics of the compositions and other pharmacologically active compounds. Examples of other excipients include solvents, surfactants, emollients, preservatives, colorants, fragrances and the like. The choice of other excipients will also depend on the form of administration used.

The form of administration used may be any suitable delivery systems considered by those skilled in the art as capable of delivering drugs to human or other animal oral cavities to achieve an anticariogenic, remineralization or reconstructive effect. Typical forms of administration include, but are not limited to, systems used to topically treat the mouth cavity and systems for ingestion.

Forms of topical administration which may be used include, but are not limited to, creams, lotions, gels, emulsions, rinses, liposomes, aerosols, oral hygiene preparations and sustained release systems. Examples include toothpaste, mouth wash breath fresheners, toothpaste, gels, and dental cavity filling compositions.

Ingestible forms of administration include, but are not limited to, dietary compositions, dietary supplements, pharmaceutical preparations and oral hygiene or health promoting preparations and delivery systems where an increase in calcium is required. Dietary compositions of particular interest are confectionary, chewing gum, breath fresheners, soft gelatin sweets, chocolate, carbonated beverages, frozen confectionary, dairy foods including yoghurt, ice cream, or other cariogenic foods or food components.

In one embodiment of this invention, the dietary composition or oral hygiene preparation further comprises an effective amount of fluorine or a fluorine containing substance for anti-dental caries.

The term “dietary composition” as used herein is generic for human or veterinary foods. Specifically, the dietary compositions of the present invention include functional foods such as fortified beverages, nutritional foods, sports bars, sports drinks; liquid and powdered drinks such as coffee, tea, juice, processed milk, and sports drinks; baked foods such as bread, pizza, biscuits, cake; pastas such as spaghetti, macaroni, wheat noodles, Chinese noodles; confectionary such as candy, gelatin confectionary, chewing gum, chocolate; frozen confectionery such as ice cream, sorbet; dairy products such as cream, cheese, powdered milk, condensed milk; yoghurt.

The term “oral hygiene preparation” as used herein refers to any composition, which can be introduced into the oral cavity and can be in contact with teeth, other than foods and drinks. The oral hygiene preparation may be drugs, herbals, plant extracts, cosmetics, vitamins, lozenges, dental floss, toothpicks, artificial saliva, mouthwash, gargle, toothpaste, dentifrices which have the effects of preventing tooth decay, whitening teeth, removing dental plaque, cleansing the oral cavity, preventing halitosis, removing plaque, or preventing deposition of dental calculus.

In a preferred form, the composition of the present invention comprises in addition to a complex of the first aspect, a hydrophilic pharmaceutically acceptable compound suitable for the treatment of caries. The term “hydrophilic pharmaceutically acceptable compound” refers to a compound which is solubilized and/or dispersible in water. The hydrophilic pharmaceutically acceptable compounds can be used alone, or in conjunction with any other substances known to those skilled in the art to have an anti-dental caries or health promoting function. These hydrophilic pharmaceutically acceptable compounds and other compounds may include but are not limited to polyphenols such as flavan-3-ol derivatives, (for example catechin, epicatechin, gallocatechin, epigallocatechin, including derivatised green tea phenolics described by Yasuda et al, extracts from molasses, fruit, coffee and chocolate), various oligosaccharides, phosphorylated oligosaccharides, fructooligosaccharides, acidic saccharides, sugar alcohols (xylitol, erythritol, palatinit, sorbitol, maltitol, mannitol, chondroitin sulfate, glucose-6-phosphate etc), organic acids (e.g., tartaric acid, citric acid, malic acid, lactic acid, fumaric acid, and maleic acid), various plant extracts (Mint oil, chamomile, ginger, rosemary, sage, etc), ascorbyl phosphate, pyridoxal 5-phosphate and vaccines. The preferred compounds in this area are those which contain an anionic moiety such as calcium ascorbyl phosphate.

The hydrophilic pharmaceutically acceptable compounds and other compounds may be in the form of a salt, such as a metal salt. Examples of a metal used for the formation of such a metal salt include alkali metal, alkaline earth metal, zinc, iron, chromium, lead, potassium, sodium, calcium, and magnesium are included. Further, the hydrophilic pharmaceutically acceptable compounds may be in the form of an ammonium salt or a quaternary amine salt.

According to a fourth aspect of the present invention, there is provided a method of treating dental caries comprising the step of administering a complex of the first aspect or a composition of the second aspect to the mouth or teeth.

Preferably, the inorganic phosphate is a calcium phosphate.

In order that the nature of the present invention may be more clearly understood, preferred forms thereof will now be described with reference to the following non-limiting examples.

### Composition Example 1

A toothpaste for use in the method of treatment or prevention of dental caries and gingivitis according to the invention was prepared as follows:

| | Ingredients | % w/w |
|---|---|---|
| A) | Sorbitol USP | 15.0 |
| | Calcium phosphate complex OD Stearamidopropyl | 7.5 |
| | dimethylamine | |
| B) | Glycerin USP 96% | 10.0 |
| | Triclosan | 0.3 |
| | Na-Saccharin USP 40/60 Mesh | 0.2 |
| | Veegum D-Granular | 2.0 |
| | Peppermint Oil | 1.1 |
| | Stepanol WA/100 (Na-Lauryl Sulfate) | 2.2 |
| C) | Veegum HF-6% (Ag/Al Silicate) | 16.64 |
| | Blue #1 FD + C (0.6%) | 0.06 |
| D) | Na-CMC 7 H 5% | 45.0 |

The components of A were combined together and then all items of B were added to A and mixed until uniform. C was then added and mixed until uniform. Finally, D was added slowly with mixing until uniform. Citric acid q.s. to pH 5.9 to 6.3

### Composition Example 2

A toothpaste of Example 1 above containing at least 0.05% Green tea extract (Sunphenon, Taiyo Kagaku Japan). The product is supplied as a slightly brown water-soluble free flowing powder and contains at least 72% polyphenols. Sunphenon is added to a hydro alcoholic solution containing 5% of the stearamidoamine complexed with calcium phosphate monobasic in a two/one mole ratio together with flavour. Food colouring q.s. to provide a mouthwash with anti cariogenic properties.

### Composition Example 3

The toothpaste of Example 1 above containing the addition of 0.3% sodium monofluorophosphate for children

### Composition Example 4

The toothpaste of Examples 2 and 3 above containing the laurimimopropionate complex ½ mole ratio with calcium fluorophosphates at 2% wt/wt.

### Composition Example 5

The toothpaste of Example 1 above containing the addition of a tooth whitening compound and 5-7% wt/wt of merquat 550 complex of calcium phosphate dibasic at 1/10 mole ratio.

### Composition Example 6

The toothpaste of Example 1 above containing the addition of a sensitizing compound and 5-7% wt/wt of merquat 550 complex of calcium phosphate dibasic at 1/10 mole ratio.

### Composition Example 7

This composition provides a dental filling material.

| | |
|---|---|
| Calcium phosphate/arginine | 5% w/w |
| Calcium phosphate | 70% |
| Acrylic polymer | 25% |
| Catalyst | trace |

### Composition Example 8

This composition provides a dental rinse comprising a hydroalcoholic solution containing 1% stearamidopropyl dimethyl ammonium/calcium superphosphate complex and 0.2% sodium fluoride.

### Composition Example 9

This composition provides a mouthwash.

| | |
|---|---|
| Calcium phosphate | 2.0 |
| Stearamidopropyl dimethylamine | 0.5 |
| Poloxamer | 1.0 |
| Flavour | q.s. |
| Water/Ethanol | q.s. ad 100% |

### Confectionary Composition Example 10

This composition provides a chewing gum.

| Ingredients | % w/w |
|---|---|
| Cane Sugar or low GI sugar citied in WO 2005/117608 | 2.0 |
| Calcium phosphate complex OD Stearamidopropyl | 7.5 |
| dimethylamine | |
| Gum Base | q.s. |
| Wheat glucose syrup | 0.5 |
| Food acid (296) | 1.0 |
| Humectant (422) | 2.0 |
| Flavour | q.s. |
| Emulsifier (322 from Soy) | 0.5 |
| Colours (100, 133) | 0.0002 |
| Antioxidant (BHT) | 0.1 |

### Confectionary Composition Example 11

The chewing gum of Example 10 above containing 0.05% green tea extract (Sunphenon, Taiyo Kagaku) and 5-7% wt/wt of merquat 550 complex of calcium phosphate dibasic at 1/10 mole ratio.

### Confectionary Composition Example 12

This composition provides a soft gelatine confectionary.

| Ingredients | % w/w |
|---|---|
| Wheat Glucose Syrup | 36% |
| Cane Sugar | 32% |
| Wheat or Corn Starch | 23% |
| Gelatine | 6% |
| Citric acid | 0.95% |
| Fruit juice concentrate | qs |
| Natural flavours | qs |
| Natural colors | qs |
| Calcium phosphate complex OD | 3.0% |
| Stearamidopropyl dimethylamine | |
| Green tea extract (Sunphenon) | 0.05% |

### Example 13

### Preparation of Arginine Calcium Glycerophosphate

Deionised water 897.6 g is charged to a vessel and heated to 60° C. to which arginine 174.2 g is added and mixed until dissolved. A molar equivalent of calcium glycerophosphate (C₃H₇CaO₆P) is dispersed into the solution and mixed until homogeneous. The mixture is cooled to 30° C. and the pH adjusted with dilute acid or base as desired, preservative is added and the product diluted to a 30% w/v aqueous slurry of the complex.

The slurry can be dried if desired by any suitable method including spray drying, freeze drying and drum drying.

### Example 14

### Preparation of Stearamidopropyl Dimethylamine Glycerophosphate

Deionized water (600 g) is heated to 70° C. with mixing. Stearamidopropyl dimethylamine 0.5 moles (184.5 grams) is added and mixed until homogeneous. The solution is cooled to 50° C. and one mole equivalent of calcium glycerophosphate is added as in example 12 above, or if desired 1 mole of calcium pyrophosphate is added and the mixture cooled with stirring to 30° C. Sufficient deionized water is added to yield a 25% wt/wt solution of complex/s. Preservative is added as needed. The pH is adjusted with 20% citric acid or 10% NAOH to obtain a pH of 4-8. Drying may be done as above if desired but is optional.

### Example 15

### Preparation of EPB-calcium Glycerophosphate Complex

21 parts by weight of calcium glycerophosphate were added to 211.5 parts deionized water and mixed at 40-50° C. until dissolved. 120 parts of the EPB (ethoxylated phosphobetaine) with 8 moles of ethylene oxide (MW 837) were added to form a smooth homogeneous slurry to which was added 10% citric acid to adjust the final ph to 5-5.5.

### Example 16

This example investigated the tooth remineralisation properties of a complex according to the invention.

Materials

BMM: basal medium mucin models the nutrients present in saliva and was prepared following Wong et al, “Calcium phosphate deposition in human dental plaque microcosm biofilms induced by a ureolytic pH-rise procedure” Archives of Oral Biology 47 (2002) 779-790

| | |
|---|---|
| PLQ7 | BMM plus calcium phosphate monofluorophosphate urea |
| | (CPMU) solution as a positive control. |
| PLQ8 | BMM plus water as a control |
| PLQ9 | BMM plus a calcium phosphate complex with Pecosil, a |
| | silicon based surfactant. The resultant complex comprised |
| | a silicon-based backbone with 112 calcium phosphate side |
| | groups. The composition contained 2% of the complex and |
| | had a pH of 7. |
| PLQ10 | BMM plus the EPB-calcium glycerophosphate complex from |
| | Example 14. The composition contained 1% of the complex |
| | and had a pH of 5. |

Methodology

The complex according to the invention was tested for its tooth remineralisation properties using the methodology described in Wong et al, “Calcium phosphate deposition in human dental plaque microcosm biofilms induced by a ureolytic pH-rise procedure” Archives of Oral Biology 47 (2002) 779-790.

Ca & P units: mmol/g protein, F units: μmol/g protein

Mineralisation regime: 14 days mineralisation; 3 doses of testing composition and 10% sucrose daily

Results

Means, SD and SE for MAM60C Ca, P, F Mineral Data-protein
basis - Total mineral (n = 4)

| | Mean | SD | SE |
|---|---|---|---|
| | Calcium (mmol/g protein) | | |
| PLQ7 (CPMU) | 0.399 | 0.081 | 0.040 |
| PLQ8 (water control) | 0.124 | 0.011 | 0.005 |
| PLQ9 (2%, pH 7) | 0.167 | 0.026 | 0.013 |
| PLQ10 (1% pH 5) | 0.298 | 0.088 | 0.044 |
| | Phosphate (mmol/g protein) | | |
| PLQ7 (CPMU) | 1.385 | 0.199 | 0.100 |
| PLQ8 (water control) | 0.996 | 0.114 | 0.057 |
| PLQ9 (2%, pH 7) | 1.396 | 0.065 | 0.032 |
| PLQ10 (1% pH 5) | 1.030 | 0.367 | 0.183 |
| | Calcium:Phosphate | | |
| PLQ7 (CPMU) | 0.298 | 0.049 | 0.025 |
| PLQ8 (water control) | 0.126 | 0.022 | 0.011 |
| PLQ9 (2%, pH 7) | 0.120 | 0.020 | 0.010 |
| PLQ10 (1% pH 5) | 0.347 | 0.254 | 0.127 |
| | Fluoride (nmol/g protein) | | |
| PLQ7 (CPMU) | 0.267 | 0.128 | 0.064 |
| PLQ8 (water control) | 0.060 | 0.006 | 0.003 |
| PLQ9 (2%, pH 7) | 0.155 | 0.060 | 0.030 |
| PLQ10 (1% pH 5) | 0.271 | 0.115 | 0.058 |
| | Calcium:Fluoride | | |
| PLQ7 (CPMU) | 1759 | 840 | 420 |
| PLQ8 (water control) | 2084 | 107 | 54 |
| PLQ9 (2%, pH 7) | 1169 | 352 | 176 |
| PLQ10 (1% pH 5) | 1237 | 547 | 273 |

Significance (Ca, P, F)

| | PLQ7 | PLQ8 | PLQ9 | PLQ10 |
|---|---|---|---|---|
| PLQ7 | | ** | ** | |
| PLQ8 | | | | ** |
| PLQ9 | | | | ** |
| PLQ10 | | | | |

** p < 0.001

### Conclusion

The results show that the complex according to the invention from Example 14 has tooth remineralisation properties.

Modifications and improvements to the invention will be readily apparent to those skilled in the art. Such modifications and improvements are intended to be within the scope of this invention.

The word ‘comprising’ and forms of the word ‘comprising’ as used in this description and in the claims does not limit the invention claimed to exclude any variants or additions.

What is claimed is:

1. A nitrogen complex of an inorganic phosphate comprising the anionic sub-structure (I) 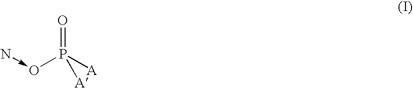
whereby a nitrogen atom (N) of a complexing agent is complexed to an oxygen atom (O) of the inorganic phosphate;
wherein the nitrogen atom is selected from the group consisting of a nitrogen atom of an amine group or a nitrogen atom of a N-heteroaromatic ring; and
wherein A and A′ are each independently selected from the group of constituents consisting of a hydroxyl group (—OH), an oxide group (—O⁻) the group —OR where R is alkyl or substituted alkyl; an oxygen to which a nitrogen is complexed (—O←N) wherein the nitrogen atom is selected from the group consisting of a nitrogen atom of an amine group or a nitrogen atom of a N-heteroaromatic ring; and a phosphate of the form 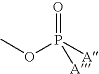
wherein each of A″ and A′″ are selected independently from the same group of substituents as A′ and A″;
wherein at least one of A, A′, A″ and A′″ is an oxide (—O⁻) with the proviso that when A″ and A′″ are not present then at least one of A and A′ is an oxide (—O⁻).

2. A complex according to claim 1 wherein the inorganic phosphate comprises a counter cation selected from the group consisting of alkali metals (Group I), alkaline earth metals (Group II) and mixtures thereof.

3. A complex according to claim I wherein the inorganic phosphate is a calcium phosphate.

4. A complex according to claim 3 wherein the calcium phosphate is selected from the group consisting of calcium phosphate monobasic (Ca(H₂PO₄)₂), calcium dibasic (Ca HPO₄), calcium phosphate tribasic (Ca₃(PO₄)₂), superphosphates of calcium, fluorinated calcium superphosphate, calcium phosphate salts either in amorphous or crystalline forms (including apatites and hydroxyapatites) and mixtures thereof.

5. A complex according to claim 1 wherein R, when present, is glycerol.

6. A complex according to claim 1 wherein the nitrogen is a nitrogen atom selected from the group consisting of primary, secondary, tertiary and quaternary amines and mixtures thereof.

7. A complex according to claim 6 wherein the nitrogen is a nitrogen atom of a tertiary amine.

8. A complex according to claim 7 wherein the tertiary amine is selected from the group consisting of stearamidopropyldimethylamine, lauryliminodipropionic acid and mixtures thereof.

9. A complex according to claim 1 wherein the nitrogen is a nitrogen atom of a compound selected from the group consisting of amino acids, proteins, polypeptides, amine functional sterols, phospholipids containing amine functional groups and mixtures thereof.

10. A complex according to claim 9 wherein the compound is arginine.

11. A complex according to claim 1 wherein the complexing agent is a water soluble polymer selected from the group consisting of amphoteric, zwitterionic and cationic surfactants, polymers having amine groups, phosphobetaines, ethoxylated phosphobetaines and mixtures thereof.

12. A complex according to claim 11 wherein the complexing agent is selected from merquats, cationic guar gums, aminated polysilicone trimethylsilylamodimethicone and mixtures thereof.

13. A method of preparing a complex according to claim 1 comprising the step of reacting a complexing agent containing nitrogen with an inorganic phosphate.

14. A composition for the administration of an inorganic phosphate comprising an effective amount of one or more complexes according to claim 1.

15. A composition according to claim 14 further comprising other compounds for the treatment of the teeth or the mouth cavity selected from the group consisting of pyrophosphates, antibacterials, pharmaceuticals, nutrients, fluoride and phosphatase inhibitors, aggregating divalent and trivalent metal ions; whitening agents, calcium phosphate monofluorophosphate urea (CPMU), substances that change oral pH, salicylamides, salicylanilides, plant extracts and oils, metal ions, anti-plaque agents, anticaries agents, pH buffering agents, anti-staining agents, bleaching agents, desensitizing agents, dyes, colors, surfactants, binders, sweeteners, humectants, abrasive agents, and mixtures thereof.

16. A composition according to claim 14 further comprising a hydrophilic pharmaceutically acceptable compound suitable for the treatment of caries.

17. A composition according to claim 16 wherein the hydrophilic pharmaceutically acceptable compound suitable for the treatment of caries is selected from the group consisting of polyphenols, oligosaccharides, phosphorylated oligosaccharides, fructooligosaccharides, acidic saccharides, sugar alcohols, organic acids, various plant extracts, ascorbyl phosphate, pyridoxal 5-phosphate and vaccines.

18. A method of treating dental caries comprising the step of administering a complex according to claim 1.

19. A method of treating dental caries comprising the step of administering a composition according to claim 14.
